(19) United States
(12) Patent Application Publication
(10) Pub. No.: US 2025/0326763 A1
(43) Pub. Date: Oct. 23, 2025

(54) MELANIN DERIVED FROM PALM POLLEN

(71) Applicant: Melanovate Technologies Inc., Carlsbad, CA (US)

(72) Inventors: Rashad Tawashi, Carlsbad, CA (US); Mona Tawashi, Carlsbad, CA (US); Salma Hassouna, Carlsbad, CA (US); Tamer Galal, Carlsbad, CA (US)

(21) Appl. No.: 19/184,780

(22) Filed: Apr. 21, 2025

Related U.S. Application Data
(60) Provisional application No. 63/637,196, filed on Apr. 22, 2024.

(51) Int. Cl. C07D 487/06; A61K 9/00; A61K 31/407; H10K 85/10; H10K 85/60

(52) U.S. Cl. CPC C07D 487/06; A61K 9/0014; A61K 31/407; H10K 85/151; H10K 85/6572

(57) ABSTRACT

Methods for extraction of melanin from date palm pollen and methods of use for the extracted melanin are described. Compositions for topical formulation that include melanin from DPP are also described, as well as methods of use of the topical formulations in treatment of skin conditions such as wound healing and treatment of vitiligo. Use of date palm pollen and melanin that is derived from date palm pollen as a bioderived organic semiconductor is also described, along with electronic devices into which the melanin semiconductor is incorporated.

### CROSS-REFERENCE TO RELATED APPLICATIONS

This application claims the benefit of U.S. Provisional Application No. 63/637,196, filed on Apr. 22, 2024, which is incorporated herein by reference in its entirety.

### FIELD OF THE INVENTION

The invention relates to date palm pollen as a delivery vehicle for melanin, and to melanin extracted from date palm pollen.

### BACKGROUND

In the last decades, melanin has received considerable attention for its photoprotective, antioxidant, antimicrobial and anti-radiation properties, and also for its application in electronics as an organic semiconductor. Melanins are common pigments in nature, and are found in many different taxa groups such as bacteria, yeasts, fungi, insects, plants, reptiles, birds, and animals.

Melanin is a dark black to brown colored hydrophobic negatively charged natural heterogeneous biopolymer (Guo, L., et al. (2023) Journal of Molecular Sciences 24:4360). Melanin's existence within a broad live spectrum from microbes to higher taxa (plants and animals) indicates its numerous functions such as photo-protection, antioxidant, thermoregulation and antimicrobial (Jalili, S., et al. (2022) Journal of Survey in Fisheries Sciences 8:140-160; Glagoleva, A. Y., et al. (2020) Frontiers in Plant Science 11:770). Melanin pigment plays a significant role in the human skin. First, it acts as a thermoregulator (by converting UV into heat); in addition, it acts as a free radical scavenger (Phiddian, E. (Oct. 20, 2022) Cosmos Magazine).

Melanin pigments are categorized into eumelanin, pheomelanin and allomelanin based on their color, basic chemical entities present in the structure, and biosynthesis pathway and enzymes. Microorganisms can utilize various substrates and synthesize different types of melanin. There are different pathways leading to the synthesis of several kinds of melanin in nature, most of which have been found in bacteria (Liu, R., et al. (2022) World Journal of Microbiology and Biotechnology 38:228). In some of the biosynthetic pathways, melanin production involves specific enzymes such as tyrosinases and lactases. In some instances, melanin formation results from genetic and/or metabolic alterations in tyrosine metabolism. Melanin biosynthesis in bacteria is subject to metabolic and genetic control (Bayram, S. (2022) Indian Journal of Microbiology 62 (3): 393-400). In plants, melanin belongs to the class of allomelanin. It is nitrogen free and is referred to as catechol melanin. Currently, obtaining melanin is a complex, time consuming and costly process, which involves either chemical synthesis, culture (from bacteria, fungi, etc.), or extraction from plants/animals (Singh, S., et al. (2021) Biotechnology Advances 53:107773). Synthetic melanin is prepared from tyrosine, using an enzymatic process or by autooxidation of 1,8-dihyroxynaphthalene. New sources of melanin and/or melanin delivery systems are needed.

### BRIEF SUMMARY OF THE INVENTION

Methods of producing melanin from date palm pollen (DPP), extracted or purified melanin from DPP, and applications of use of melanin-containing DPP or melanin derived from DPP are provided herein.

In one aspect, methods for producing melanin are provided. The methods include extracting melanin from date palm pollen (DPP) of a species of the genus Phoenix, such as Phoenix dactylefera L.

In some embodiments, extraction of melanin includes ultrasound-assisted extraction of melanin from the DPP.

In some embodiments, extraction of melanin includes: (a) contacting DPP with a strong alkali solution, thereby producing an alkaline solution that includes solubilized melanin; (b) separating alkali soluble melanin from non-soluble components of the DPP; (c) contacting the alkali soluble melanin with a strong acid solution, thereby precipitating the melanin; and (d) treating the precipitated melanin with one or more organic solvent(s) to remove lipids and other residue, thereby producing extracted melanin. In some embodiments, step (a) includes solubilizing the melanin in a strong base, such as but not limited to about 2M NaOH. In some embodiments, step (b) includes centrifuging the alkaline solution produced in step (a), thereby producing a supernatant and a pellet, wherein the supernatant includes the alkali soluble melanin. In some embodiments, step (c) includes reducing the pH to about 2 with a strong acid, such as but not limited to HCl. In some embodiments, the organic solvent(s) in step (d) include chloroform, ether and/or petroleum ether. In some embodiments, the amount of melanin that is extracted from the DPP is at least about 5 times the amount of melanin that is extracted from an equivalent weight of date fruit from the same species, e.g., at least about 5 times the amount of melanin by weight is contained in an aqueous alkaline extract from DPP than in an aqueous alkaline extract of date fruit of the same species which melanin is extracted in an equivalent method, such as a method described herein, and/or at least about 5 times as much melanin by weight is purified from DPP than from date fruit of the same species from which melanin is purified in an equivalent method, such as a method described herein.

In another aspect, an aqueous melanin-containing alkaline extract of DPP is provided.

In another aspect, purified melanin is provided. The melanin may be purified according to any of the methods described above.

In another aspect, a composition is provided that is formulated in an anhydrous vehicle for topical application to an individual. The composition includes an aqueous melanin-containing alkaline extract of DPP or purified melanin as described above. In some embodiments, the melanin exhibits photoprotective, antioxidant, antimicrobial, and/or anti-radiation properties when applied to the skin of an individual.

In another aspect, a method for treating a wound is provided. The method includes administering a composition that is formulated for topical application as described herein, which includes DPP or an aqueous melanin-containing alkaline extract of DPP or purified melanin, formulated in an anhydrous or hydrous vehicle for topical application, to a skin wound in an individual in need thereof, wherein healing of the wound is accelerated by the composition.

In another aspect, a method for improving the appearance of vitiligo is provided. The method includes administering a composition that is formulated for topical application as described herein, which includes DPP or an aqueous melanin-containing alkaline extract of DPP or purified melanin, formulated in an anhydrous or hydrous vehicle for topical application, to skin of an individual who has lost pigmentation, wherein administering the composition results in at least partial repigmentation of the skin to which the composition is applied.

In another aspect, an organic semiconductor is provided, which includes DPP from a species of the genus Phoenix or melanin that is purified from the DPP. In some embodiments, the DPP is from Phoenix dactylefera L.

In another aspect, an electronic device, e.g., a biofriendly (environmentally friendly or sustainable) melanin based electronic device, is provided, which includes an organic semiconductor as described herein, i.e., including DPP from a species of the genus Phoenix or melanin that is purified from the DPP.

In another aspect, a medical sensor is provided, which includes an organic semiconductor as described herein, i.e., including DPP from a species of the genus Phoenix or melanin that is purified from the DPP. In certain embodiments, the medical sensor is incorporated into an e-skin device.

In another aspect, a semiconductor wafer is provided, which includes an organic semiconductor as described herein, i.e., including DPP from a species of the genus Phoenix or melanin that is purified from the DPP, blended with one or more polymer. In some embodiments, the polymer includes polydimethyl siloxane or poly(2,4-ethylenedioxythiophene) polystyrene sulfonate (PEDOT:PSS). In some embodiments, a solid solution of melanin purified from DPP is provided in microcrystalline cellulose to produce a composite organic semiconductor, or the melanin extracted from DPP is used to provide a thin film by evaporation under vacuum (Yang, Y. B., et al. (2025) Small 21 (9): e2410159).

### BRIEF DESCRIPTION OF THE DRAWINGS

FIG. 1 shows a Fourier Transform Infrared Spectrum (FTIR) of date palm pollen.

FIG. 2 shows a FTIR spectrum of melanin extracted from DPP as described herein. The FTIR spectra are in agreement with the FTIR spectrum of the melanin extracted from date fruit. (Alam, M. Z., et al. (2022) Scientific Reports 12:6614; Alam, M. Z., et al. (2024) Scientific Reports 14:4683)

FIGS. 3A-3E show microscopic and cytochemical imaging of DPP. FIGS. 3A-3B: optical and scanning electron microscope images, respectively, of dry DPP powder. FIG. 3C: fluorescent imaging of DPP. FIG. 3D: staining with ferric chloride and interaction of the ferric ion with the DPP surface wall. FIG. 3E: interaction of (6,7-dimethoxy-4-coumaryl) alanine (DMCA) stain with the external wall (exine) of the DPP cell wall.

FIGS. 4A-4C show the particle size distribution of DPP obtained from image analysis. FIG. 4A: DPP fluorescent microscope (dry). FIG. 4B: DPP 4-(dimethylamino)-cinnamaldehyde (DMACA) stain (wet). FIG. 4C: DPP ferric chloride/ferric ferrocyanide (wet).

### DETAILED DESCRIPTION

As described herein, the inventors have confirmed the presence of melanin in DPP. Provided herein are applications of use for melanin in date palm pollen (DPP), and methods for extraction and purification of melanin from DPP. Applications of use of melanin from DPP in cosmetic and dermatological formulations and as sustainable organic semiconductors are provided.

### Definitions

Unless defined otherwise herein, all technical and scientific terms used herein have the same meaning as commonly understood by one of ordinary skill in the art to which this invention belongs. Singleton, et al., Dictionary of Microbiology and Molecular Biology, second ed., John Wiley and Sons, New York (1994), and Hale & Markham, The Harper Collins Dictionary of Biology, Harper Perennial, NY (1991) provide one of skill with a general dictionary of many of the terms used in this invention. Any methods and materials similar or equivalent to those described herein can be used in the practice or testing of the present invention.

Numeric ranges provided herein are inclusive of the numbers defining the range.

“A,” “an” and “the” include plural references unless the context clearly dictates otherwise.

The term “about” is used herein to mean plus or minus ten percent (10%) of a value. For example, “about 100” refers to any number between 90 and 110.

The terms “and/or” and “or” are used interchangeably herein and refer to a specific disclosure of each of the two specified features of components with or without the other. Thus, the term “and/or” as used in a phrase such as “A and/or B” herein is intended to include “A and B,” “A or B,” “A” (alone), and “B” (alone). Likewise, the term “and/or: as used in a phrase such as “A, B and/or C” is intended to encompass each of the following aspects: A, B and C; A, B or C; A or C; A or B; B or C; A and C; A and B; B and C; A (alone); B (alone); and C (alone.

The term “antimicrobial” refers to any agent or combination of agents which is intended to kill, inactivate or inhibit the growth of any microbes such as bacteria, fungi, viruses, yeast, mold, and the like. The terms “antimicrobial” and “biocidal” are used interchangeably herein.

The term “broad spectrum” refers to an antimicrobial substance that acts against a wide range of microorganisms, for example, Gram-positive bacteria, Gram-negative bacteria, fungi, etc.

The terms “DPP,” “date palm pollen,” or “palm pollen,” used interchangeably herein, refer to pollen from a species of the genus Phoenix in the family Palmae. The term “date palm” as used herein includes both ornamental and fruit-producing species in the genus Phoenix. As used herein, the term an “extract” of DPP refers to material that has been extracted from pollen from a species of the genus Phoenix, or one or more compound(s) that is derived or extracted from DPP, such as, but not limited to, flavonoid(s), quercetin, triterpene(s), and/or other constituent compound(s) of DPP.

The term “derived from” encompasses the terms “originated from,” “obtained from,” “obtainable from,” “isolated from,” “extracted from,” and “created from,” and generally indicates that one specified material finds its origin in another specified material or has features that can be described with reference to another specified material.

“Effective amount” as used herein refers to an amount (e.g., minimum inhibitory concentration (MIC)) of a preservative composition as disclosed herein that is sufficient to prevent or inhibit microbial growth. The preservative compositions described herein may be active against Gram-positive bacteria, Gram-negative bacteria, yeast, fungi, and/or molds.

An “individual” refers to a mammal, often a human.

The terms “microorganism” and “microbe” are used interchangeably herein and refer to microscopic organisms, e.g., microscopic single celled life forms, including but not limited to bacteria, fungi, and algae microorganisms. Microbes may exist in single-celled form or in a colony of cells or in a biofilm. Microbes include eukaryotes and prokaryotes. Nonlimiting examples of microbes include bacteria, fungi, algae, archaea, protozoa, viruses, and the like.

The term “pharmaceutical” refers to a substance, e.g., a chemical substance, which may be used in medical treatment, cure, or prevention of a condition for which administration of the substance is beneficial, such as a disease condition.

The term “preservative” refers to a substance or agent that is added to a product to prevent decomposition by microbial growth and/or by undesirable chemical changes.

The terms “recovered,” “isolated,” “purified,” “extracted,” and “separated” as used herein refer to a material (e.g., a protein, nucleic acid, cell, or small molecule) that is removed from at least one component with which it is naturally associated. For example, these terms may refer to a material which is substantially or essentially free from one or more components which normally accompany it as found in its native state, such as, for example, an intact biological system, such as, for example, intact DPP.

A “semiconductor” as used herein refers to a material that has a conductivity between a conductor, such as a metal, and a nonconductor or insulator, such as a ceramic.

The term “shelf life” refers to the length of time for which an item (e.g., a product as described herein) remains usable, fit for consumption, or saleable, e.g., free from microbial contamination, color changes, etc.

The term “topical formulation” as used herein refers to a composition that is formulated for application to an external body surface, such as skin or mucous membranes.

“Unit dose” refers to an amount of a substance contained in a formulation for topical administration as described herein that is sufficient to cause a therapeutic, prophylactic, or cosmetic effect when applied to an external body surface of an individual. A unit dose may be administered in a single topical application of the formulation or in two or more applications.

A “wafer” as used herein refers to a thin slice of semiconductor material that is used in electronics for construction of integrated circuits.

### Date Palm Pollen

The compositions and methods described herein include pollen from a palm in the genus Phoenix and/or a melanin-containing aqueous alkaline extract thereof. In some embodiments, pollen from a date palm is used. In one exemplary embodiment, the formulations herein include pollen from the species Phoenix dactylifera L, Palmae. The pollen can be harvested from male date palm trees during the flowering season in the months of February and March.

Pollen grains have been designed by nature to transfer genetic material from plant-to-plant. Date palm pollen (DPP) exists in a very fine powder material, produced by the male flowering date palm. DPP is the male reproductive cell of the date palm. The male flower develops 2-3 weeks before the female flower. Once the male pods open, they may be removed from the tree and dried. Once dried, pollen may be stored in a cool environment. DPP represents the reproductive cell of the male flower and contains the male contribution to the next generation of the plant.

DPP applications and uses in traditional and herbal medicine have been recorded throughout history. The early Egyptians used DPP as a rejuvenating medicinal agent; oral suspension of DPP as an herbal mixture is still widely used as a folk remedy for the treatment of male infertility. Recently, date palm fruits were found to contain melanin at a concentration of 1.5 to 5%. The melanin is localized in the tanniferous cells between the inner and outer mesocarp tissues of the date fruit (Alam, M. Z., et al. (2022) Scientific Reports 12:6614). However, melanin has not been previously reported to be present in date palm pollen grains.

### Melanin

Melanin's role in skin is twofold. Firstly, it acts as a thermoregulator by converting light energy (UV) into heat. In addition, it acts as a free radical scavenger, dissipating free radicals. For this reason, DPP containing melanin will act as a photoprotector, radiation protector, antioxidant, and antimicrobial agent in cosmetic or pharmaceutical formulations, such as formulations for topical administration.

Example 1 describes a study that confirmed the presence of melanin in DPP. FTIR analysis confirmed the presence of melanin. Microscopic and cytochemical analysis support the FTIR data. The behavior of DPP was studied in hydrophilic and hydrophobic media using image analysis. These studies suggest that DPP is a sustainable, biocompatible, biodegradable and economic source for melanin. Melanin-containing DPP provides a new organic mixed ionic electronic conductor for sustainable electronics. The particle size, shape and surface characteristics of DPP and the economic availability of melanin naturally carried in this tiny microcapsule offers large-scale applications and opportunities.

The free flowing properties of naturally melanin loaded DPP powder can be easily dispersed homogenously in liquids, semi-solids and solid preparations. There is no requirement for cultures or chemical synthesis that is involved in the current production of melanin. DPP microcapsules are an ideal new delivery system for melanin in cosmetic and dermal pharmaceuticals. DPP also may be utilized in applications in the electronics/biomedical field as an environmentally friendly organic semiconductor.

DPP offers unique opportunities to the pharmaceutical and cosmetic formulator. The free flowing fine powder that has a consistent particle size distribution and carries eumelanin in microcapsular form provides a new pharmaceutical and cosmetic ingredient. Large-scale production of melanin with consistent structural properties for commercial needs has previously been complex and costly. DPP microcapsules carrying melanin provide a new economical and sustainable source. Moreover, its biocompatibility, ability to disperse homogenously in other components (plastics) provides applications in biomedical, dermopharmaceutical, cosmetic formulations, and medical electronics (Nozella, N. L., et al. (2023) Materials Advances 4:4732-4743; Michael, H. S. R., et al. (2023) Archives of Microbiology 205:306; Contreras-Pereda, N., et al. (2024) Materials Today Chemistry 35:101855). Instead of synthesizing melanin-like polymers for medical devices such as e-skin devices, DPP containing melanin can be developed into a biocompatible, economic multifunctional platform for medical sensors.

As described herein, melanin may be extracted from DPP under aqueous alkaline conditions. Aqueous alkaline melanin-containing extracts of DPP are provided. Melanin that has been precipitated by neutralization and/or acidification of an aqueous alkaline melanin-containing extract of DPP is also provided. Melanin may also be purified from an alkaline extract of DPP, for example, by removing components such as lipids with one or more solvent(s), such as but not limited to chloroform, ether, and/or petroleum ether. Purified melanin from DPP is provided, for example, melanin that is at least partially purified from an aqueous alkaline extract of DPP.

### Methods of Producing Melanin

As described herein, melanin may be extracted from DPP, such as DPP from a species of the genus Phoenix, e.g., Phoenix dactylefera L. Extraction of melanin from DPP may include ultrasound-assisted extraction (passing ultrasonic energy through a liquid that contains DPP) and/or extraction under aqueous alkaline conditions. In some embodiments, ultrasound serves as a cell disruptor and facilitates the extraction of melanin from the DPP cell wall.

In some embodiments, the method includes contacting DPP with a strong alkali solution, thereby solubilizing melanin in the DPP. In certain embodiments, the strong alkali solution may be a solution (e.g., about 1.5 M to about 2.5M) of a strong base, such as but not limited to sodium hydroxide or potassium hydroxide. In some embodiments, the pH for base extraction is about 13 to about 14. Alkali soluble melanin may be separated from non-soluble components of DPP, such as cell walls or other insoluble cellular components or cellular debris, such as by centrifugation or other separation technique. Alkali soluble melanin in the supernatant may then be contacted with an acid, thereby precipitating melanin. In certain embodiments, a strong acid solution is used, such as but not limited to hydrochloric acid for example, reducing the pH to about 1.5 to about 3. Optionally, at least a portion of non-melanin components of the precipitate may be removed. For example, the precipitate may be contacted with one or more organic solvent(s) to remove lipids or other residue, such as but not limited to chloroform, ether, and/or petroleum ether.

Quantitative gravimetric analysis of melanin separated from DPP by the methods described herein showed that DPP contains about 15% (w/w) melanin. This concentration is about 5 times the reported concentration of melanin in date fruit (1.5-5%). This finding indicates that DPP is a valuable source of melanin for extraction and commercialization. Melanin separated from DPP by the methods described above was confirmed by FTIR and UV-vis spectrum analysis.

### Compositions

Pharmaceutical or cosmetic compositions are provided, which include DPP or extracted or purified melanin from DPP as described herein, e.g., melanin prepared by aqueous alkaline extraction of DPP. For example, the melanin-containing composition may be formulated for topical administration to the skin of an individual. Melanin in the composition may provide photoprotective, antioxidant, antimicrobial, and/or anti-radiation properties to the composition in which it is formulated and/or to the individual to whom it is administered.

Melanin-containing compositions as described herein may be formulated in an anhydrous or hydrous (aqueous) vehicle for topical administration. In some embodiments, the melanin-containing composition is formulated for topical administration for treatment of a skin wound in an individual. In some embodiments, the melanin-containing composition is formulated for treatment, e.g., improvement of the appearance, of vitiligo.

Compositions are provided that include DPP or extracted or purified melanin from DPP as described above, formulated for topical application to an external body surface. A composition herein may be formulated in any convenient form for topical administration. The form and components to be included in the composition will depend on the site of administration and intended use of the composition, as will be readily apparent to those of skill in the art. For example, the composition may be formulated as a powder, a compact disc, a suspension, a cream, a lotion, an ointment, a gel, a foam, a paste, an aerosol, a body wash, a hair product, a sunscreen, a lipstick, an emulsion, a cosmetic product, an anhydrous absorption base composition, or any composition that may be applied to an external body surface, for application of DPP or extracted or purified melanin from DPP.

In some embodiments, a composition is formulated for delivery of a unit dose of melanin to an individual. A unit dose for application to a human face may contain, for example, about 1.5% to about 4.5% melanin (w/w), or about 15 mg to about 45 mg of melanin from DPP (e.g., in a 1 g dose). For application to another external body surface, a unit dose may be determined by the ratio of the surface area of the body surface to the surface area of an average human face.

Typically, in addition to the melanin, a composition herein includes one or more additional constituents to facilitate topical application. In some embodiments, one or more flow regulating agent(s) is included. For example, silica (e.g., colloidal silica), silica gel, magnesium trisilicate, or cornstarch may be included as a flow regulating agent. A flow regulating agent may be included to increase flow of the formulation, depending on the application of use. A powder formulation or compact disc formulation may include one or more such flow regulating agent(s). A compact disc formulation may further include one or more filler(s), such as titanium dioxide, spray dried lactose, or starch, and may optionally further include one or more excipient(s) such as magnesium stearate to facilitate compression.

In some embodiments, the composition is formulated as a suspension, and in addition to DPP or extracted or purified melanin from DPP, the suspension may contain one or more alcohol(s) Optionally, one or more preservative substance(s) may be included for storage and shelf life stability, such as, for example, benzalkonium chloride, benzyl alcohol, or parabens. Optionally, one or more suspending(s) agent may be included, for example, carboxymethyl cellulose or xanthan gum.

In some embodiments, the composition is formulated as a cream. In one embodiment, the cream is a vanishing cream formulation. A cream formulation may contain, in addition to DPP or extracted or purified melanin from DPP, one or more surfactant(s), an oil phase, an aqueous phase, one or more preservative(s), and one or more stabilizer(s). A cream formulation may include one or more surfactants selected from, but not limited to, sorbitan monostearate, polysorbate 60, and polysorbate 20, one or more substance(s) forming and/or contained within the oil phase selected from, but not limited to, stearic acid, cetyl alcohol, isopropyl palmitate, isopropyl alcohol, glycerine, and polyethylene alcohol, a water-based aqueous phase containing substances in the formulation that are soluble in water and/or that segregate to the aqueous phase, one or more preservative(s) selected from, but not limited to, methylparaben, propylparaben, and benzyl alcohol, and one or more stabilizer(s) selected from, but not limited to, sorbitol, glycerine, and glyceryl laurate.

In some embodiments, the composition is formulated as a reconstituted milk suspension. A reconstituted milk suspension may contain extracted or purified melanin from DPP and a flow regulating agent, such as colloidal silica, which may be included as a dispersing agent. The melanin and flow regulating agent may be reconstituted in milk (e.g., homogenized and/or pasteurized milk).

In some embodiments, the composition is formulated as an anhydrous absorption base composition. For example, a liquid or semi-liquid anhydrous absorption base composition may contain, in addition to extracted or purified melanin from DPP, one or more humectant(s) (one or more substance(s) that seals moisture in skin, e.g., act as moisturizers), for example, coconut oil, almond oil, and/or lanolin; one or more surfactant(s) or emulsifier(s), for example, lecithin; and one or more consistency regulator(s) or thickener(s), for example, beeswax. For example, a cream (e.g., a thick cream) anhydrous absorption base composition may contain, in addition to the melanin, one or more humectant(s), for example, coconut oil and/or lanolin; one or more surfactant(s) or emulsifiers, for example, lecithin; and one or more consistency regulator(s) or thickener(s), for example, beeswax. For example, a solid anhydrous base composition may contain, in addition to the melanin, one or more humectant(s), for example, coconut oil; and one or more consistency regulator(s) or thickener(s), for example, beeswax.

Other formulations may be prepared with other components and ratios of components by a person of skill in the formulation art and as adapted to have desired properties for a particular application of use. The formulation, manufacturing, and control of topical formulations such as creams, suspensions, sprays, etc. are known to pharmacists and others in the formulation art and are described in reference texts, such as Lachman L, Leiberman H, and Kanig J “The Theory and Practice of Industrial Pharmacy,” published by Lea & Febiger (1986). Ratios of components in the formulations described herein may be adjusted by a person of skill in the formulation art based on optimum amounts necessary to provide maximum flow and stability.

### Methods of Use

A composition containing extracted or purified melanin from DPP, or DPP (i.e., melanin containing DPP from which melanin has not been extracted) as described above, may be topically applied to an external body surface of an individual in a method in which the composition provides a beneficial effect to the individual. In some embodiments, a composition as described above may be used in a method for treatment of a wound or for improvement of the appearance of vitiligo (e.g., for visual improvement of the appearance of skin). In other embodiments, a composition as described above may be used in a method for treatment of a skin condition, such as eczema, uticaria, acne, or rosacea.

A formulation containing a unit dose of melanin may be applied topically once per day or more than once per day (e.g., 2, 3, or more times per day). A unit dose may contain, for example, about 1.5% (w/w) to about 4.5% melanin (w/w), or about 15 mg to about 45 mg of melanin from DPP (e.g., in a 1 g dose).

A formulation as described herein may be applied topically, e.g., to a skin surface, and either left on or removed after a period of time. In some embodiments, the formulation is applied topically to a skin surface, and left on the skin surface.

A method for treating a wound is provided, e.g., use of extracted or purified melanin from DPP, or DPP (i.e., melanin containing DPP from which melanin has not been extracted), for healing of a skin wound. A topical formulation containing extracted or purified melanin from DPP, or DPP, e.g., a unit dose of melanin, is administered to a skin surface of an individual that contains a wound, and healing of the wound is accelerated versus healing of the wound in the absence of the melanin-containing composition. Use of extracted or purified melanin from DPP, or DPP, for wound healing is also provided.

A method for improving the appearance of vitiligo is provided, e.g., use of extracted or purified melanin from DPP, or DPP (i.e., melanin containing DPP from which melanin has not been extracted), for visual improvement of the appearance of vitiligo. A topical formulation containing extracted or purified melanin from DPP, or DPP, e.g., a unit dose of melanin, is administered to a skin surface of an individual that exhibits vitiligo, and improvement of the appearance of the vitiligo, e.g., at least partial repigmentation of skin that has lost pigmentation results.

A method for treatment of a skin condition is provided, such as but not limited to eczema, uticaria, acne, or rosacea, e.g., use of extracted or purified melanin from DPP, or DPP (i.e., melanin containing DPP from which melanin has not been extracted), for treatment of the skin condition. A topical formulation containing extracted or purified melanin from DPP, or DPP, e.g., a unit dose of melanin, is administered to a skin surface of an individual that exhibits the skin condition, and improvement of the skin condition results (amelioration, reduction in at least one symptom, or elimination of the skin condition).

### Semiconductors and Electronic Devices

DPP or melanin that is derived from DPP as described herein may be used as a bioderived, sustainable, environmentally friendly (“biofriendly)” organic semiconductor. The semiconductor (DPP, or extracted or purified melanin from DPP) may be incorporated into an electronic device, such as but not limited to an electronic medical device. The semiconductor (DPP, or extracted or purified melanin from DPP) may be incorporated into a sensor, such as but not limited to a medical sensor, such as a sensor in an electronic medical device. In one embodiment, the medical sensor is incorporated into an e-skin device. In some embodiments, DPP, or extracted or purified melanin from DPP, or a medical sensor that contains the DPP or extracted or purified melanin from DPP, is incorporated into an adhesive skin device that is used in healthcare monitoring or treatment (such as but not limited to vital signs, temperature, electromyography signals, and/or electrical stimulation) for humans or animals.

DPP or melanin that is derived from DPP as described herein may be incorporated as a semiconductor into a wafer for incorporation into an electronic device. The wafer may include the melanin semiconductor (DPP, or melanin that is derived from DPP (extracted or purified melanin from DPP)) blended with one or more polymer. In some embodiments, the wafer contains about 10% (w/w) to about 30% (w/w) DPP or about 1.5% (w/w) to about 5% melanin (w/w), blended with the polymer(s). Nonlimiting examples of polymers that may be blended with the melanin semiconductor include polydimethyl siloxane and poly(2,4-ethylenedioxythiophene) polystyrene sulfonate (PEDOT:PSS).

### EXAMPLES

The following examples are intended to illustrate, but not limit, the invention.

### Example 1

### Material and Methods

Date palm pollen (Phoenix dactylifera L) was obtained from Egypt.

### Fourier-Transform Infrared Spectroscopy (FTIR)

FTIR analysis of purified DPP was performed in a Perkin Elmer FTIR spectrometer and the DPP samples were scanned at wave length between 4000 and 400 cm⁻¹, and infrared spectrum was recorded.

### Microscopic and Image Analysis

Date palm pollen was analyzed microscopically using optical and fluorescent microscopy and Scanning Electron Microscope.

### Pollen Grain Staining

Pollen grains were stained for 40 minutes in 4-dimethylamino cinnamaldehyde (DMACA) in ethanol (0.3% w/v) and 6 N HCl (1/1: v/v). Pollen grains were then rinsed 5 times with 70% ethanol, each time for 10 minutes. Pollen grains were also stained for 40 minutes with a solution of 3% ferric chloride and 1% potassium ferricyanide and then rinsed 5 times with double-distilled water (ddH₂O). Pollen grains were then mounted between a slide and a coverslip ahead of microscopic observations. Unstained pollen grains were used as a negative control.

### Imaging

Samples were imaged with a Zeiss AxioImager Z1 equipped with an AxioCam MRm Rev.2 and an AxioCam MRmc cameras. Images were taken in brightfield, dark field, and fluorescence modes. The following objectives were used: 20× EC Plan NeoFluar, NA=0.5, 40× EC Plan NeoFluar, NA=0.75 and 63× Plan Apochromat oil, NA=1.4. In fluorescent mode, the following filter sets were used: Blue FS 49 (Excitation G 365, Emission BP445/50), Green FS 38HE (Excitation: BP 470/40, Emission BP 525/50) and Orange FS 00 (Excitation BP 557/55, Emission LP 615). Images were acquired with Zen software.

### Results

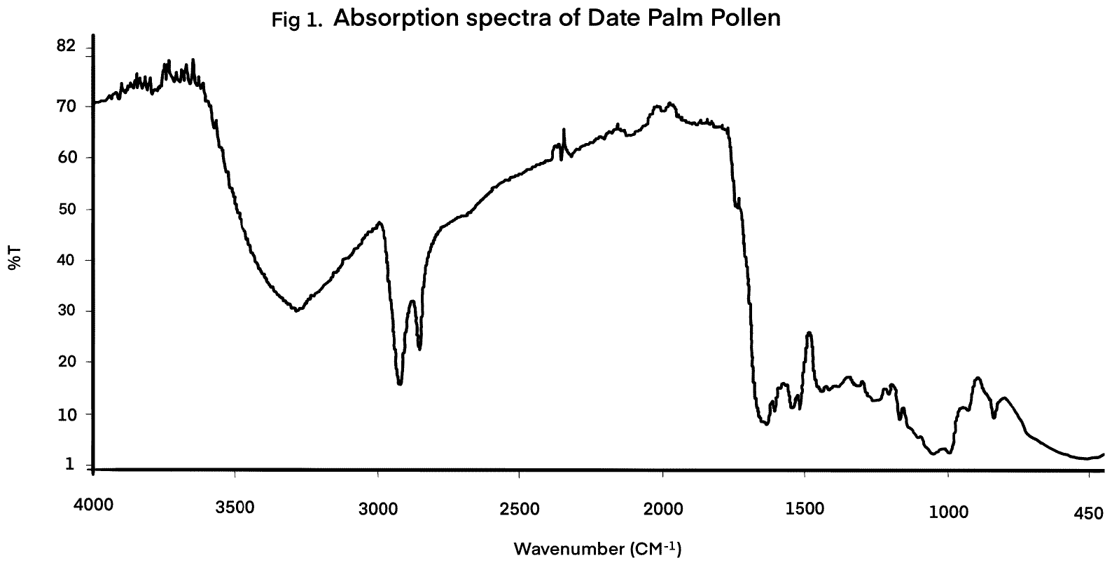

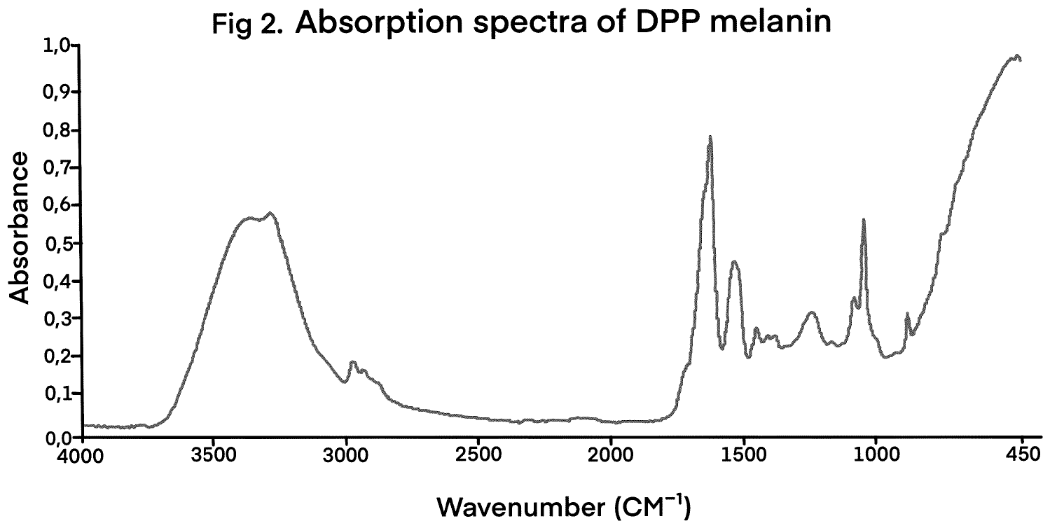

Data obtained on FTIR analysis for DPP and melanin extracted from DPP are shown in FIG. 1 and FIG. 2, respectively. The broad band absorption at approximately 3235-3281 cm⁻¹ corresponds to the stretching vibration of OH groups. The peaks observed in the range 2913-2915 cm⁻¹ were assigned to the stretching vibration of the CH groups. The characteristic band at 1602 cm⁻¹ corresponds to the vibrations of the aromatic ring C═C or symmetric stretching of COO groups. The band at 1432-14237 cm⁻¹ represents the CH₂—CH₃ ending and the peak at 1010-1255 cm⁻¹ indicates the vibration of C═O. The melanin in DPP is primarily based on (−)-epicatechin proanthocyanidin oligomers.

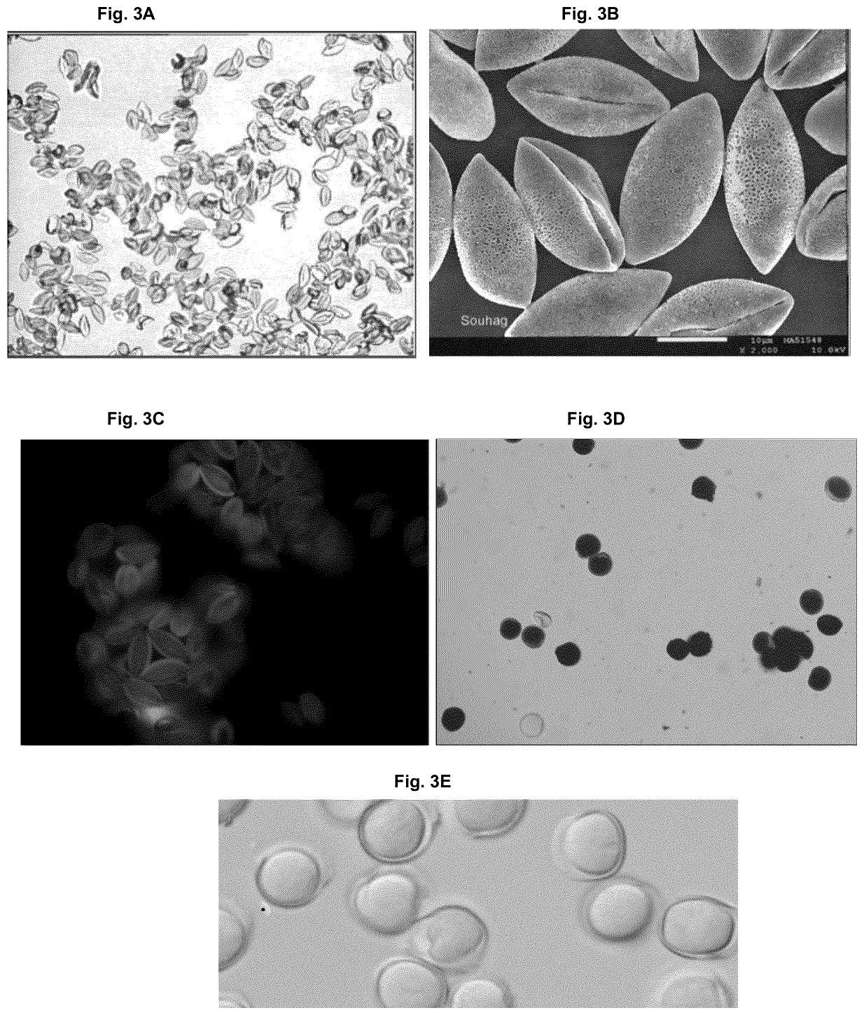

FIGS. 3A-3E show the microscopic imaging and the cytochemical study of DPP. FIGS. 3A-3B show optical and scanning electron microscope images of dry DPP powder. The images demonstrate the morphic features (size and shape) of the date palm pollen microcapsule. FIG. 3C shows fluorescent imaging of DPP. The UV-visible absorption spectrum of melanin has been mainly used to identify and characterize the pigment. Absorption at wavelengths from 230 to 500 nm has been observed for synthetic melanin and for melanin from DPP. The high level of autofluorescence observed in FIG. 3C provides additional evidence for the presence of melanin in DPP. FIG. 3D shows the staining of DPP with ferric chloride and interaction of the ferric ion with the DPP surface wall. The dark staining produced on DPP shows the interaction between the ferric ion and the phenolic group of melanin. FIG. 3E shows the interaction of the DMCA stain with the external wall (exine) of the DPP cell wall. The presence of melanin on the exine of the DPP cell may explain the presence of melanin on the mesocarp of the date palm fruit. This suggests that melanin in the date palm fruit may have originated from melanin present in the exine wall of the date palm pollen cell.

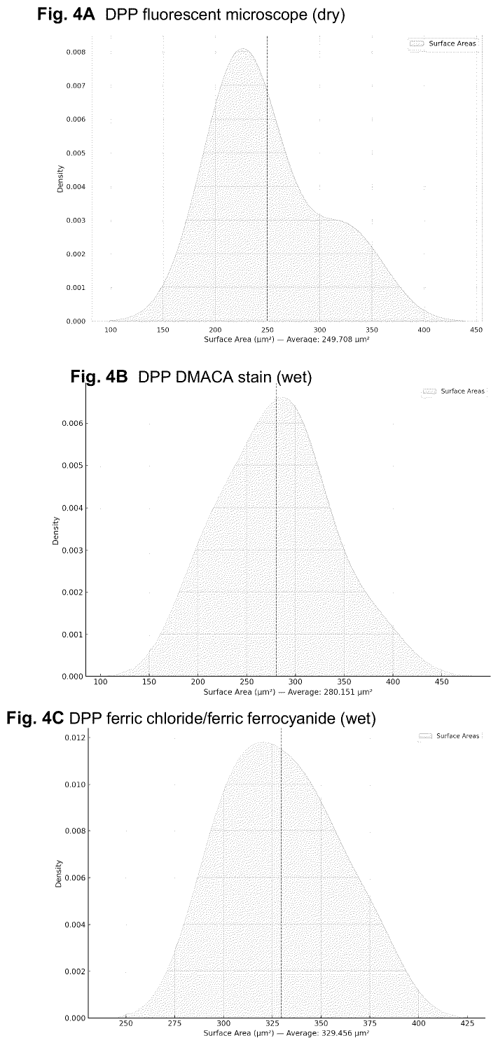

FIGS. 4A-4C show the particle size distribution of DPP obtained from image analysis. The fluorescent imaging was carried out in dry conditions (FIG. 4A), while the ferric chloride (FIG. 4C) and DMACA (FIG. 4B) imaging were carried out in wet (aqueous) conditions. A significant increase was observed in the average surface diameter of the DPP particle. This increase can reach 32% in the average particle surface diameter. The presence of moisture resulted in swelling of the DPP particle and consequently, a transformation from an elliptical shape to a nearly spherical shape, as well as an increase in the total surface area. This observation has a significant importance in the application of DPP containing melanin. For example, the significant increase in surface area means more coverage and more uniform distribution of the particle on a solid surface, e.g., change from elliptical to spherical results in a better grip on the skin surface.

The quantitative gravimetric analysis of DPP showed that DPP is a valuable source of melanin. Concentration of 15% melanin (w/w) makes DPP a significant alternative commercial source for melanin.

Although the foregoing invention has been described in some detail by way of illustration and examples for purposes of clarity of understanding, it will be apparent to those skilled in the art that certain changes and modifications may be practiced without departing from the spirit and scope of the invention, which is delineated in the appended claims. Therefore, the description should not be construed as limiting the scope of the invention.

All publications, patents, and patent applications cited herein are hereby incorporated by reference in their entireties for all purposes and to the same extent as if each individual publication, patent, or patent application were specifically and individually indicated to be so incorporated by reference.

### REFERENCES

Abdel-Shaheed, M. M., Abdalla, E. S., Khalil, A. F., & El-Hadidy, E. M. (2021). Effect of Egyptian Date Palm Pollen (Phoenix dactylifera L.) and Its Hydroethanolic Extracts on Serum Glucose and Lipid Profiles in Induced Diabetic Rats. Food and Nutrition Sciences 12 (2), 147-161.
Alam, M. Z., Ramachandran, T., Antony, A., Hamed, F., Ayyash, M., & Kamal-Eldin, A. (2022). Melanin is a plenteous bioactive phenolic compound in date fruits (Phoenix dactylifera L.). Scientific Reports 12:6614.
Alam, M. Z., Okonkwo, C. E., Cachaneski-Lopes, J. P., et al. (2024) Date fruit melanin is primarily based on (−)-epicatechin proanthocyanidin oliogomers. Scientific Reports 12:4863
Ammanagi, A., Shivasharana, C. T., Krishnaveni, R., Badiger, A., & Ramaraj, V. (2021). Functional and Structural Characterization of Melanin from Brevibacillus invocatus Strain IBA. Doklady Biological Sciences, 500 (1), 159-169
Argenziano, R., Alfieri, M. L., Gallucci, N., D'Errico, G., Panzella, L., & Napolitano, A. (2023). A model eumelanin from 5,6-dihydroxyindole-2-carboxybutanamide combining remarkable antioxidant and photoprotective properties with a favourable solubility profile for dermo-cosmetic applications. International Journal of Molecular Sciences 24: 4241
Bayram, S. (2022). A comparative characterization study between fungal and bacterial eumelanin pigments. Indian Journal of Microbiology 62 (3): 393-400.
Contreras-Pereda, N., Suárez-García, S., Pfattner, R., & Ruiz-Molina D. Melanin-inspired conductive thin films for multimodal-sensing wearable on-skin electronics. (2024) Materials Today Chemistry 35:101855.
Elkerm, Y., & Tawashi, R. (2014). Date Palm Pollen as a Preventative Intervention in Radiation- and Chemotherapy-Induced Oral Mucositis: A Pilot Study. Integrative Cancer Therapies 13:68-472.
El-Naggar, N. E. A., & El-Ewasy, S. M. (2017). Bioproduction, characterization, anticancer and antioxidant activities of extracellular melanin pigment produced by newly isolated microbial cell factories Streptomyces glaucescens NEAE-H. Scientific Reports 7, Article 42129
Eskandari, S., & Etemadifar, Z. (2021). Biocompatibility and radioprotection by newly characterized melanin pigment and its production from Dietzia schimae NM3 in optimized whey medium by response surface methodology. Annals of Microbiology 71:17.
Guo, L., Li, W., Gu, Z., Wang, L., Guo, L., Ma, S., Li, C., Sun, J., Han, B., & Chang, J. (2023). Recent advances and progress on melanin: From source to application. International Journal of Molecular Sciences 24:4360.
Ghadge, V. A., Singh, S., Doniya, E. M., Dhimar, A., Sahastrabudhe, A. G., Nimse, S. B., and Shinde, P. B. (2023) “Extraction, Purification, and Characterization of Microbial Melanin Pigments,” in Melanins: Functions, Biotechnological Production, and Applications, Springer, Cham. 29, April 291-110.
Glagoleva, A. Y., Shoeva, O. Y., & Khlestkina, E. K. (2020). Melanin Pigment in Plants: Current Knowledge and Future Perspectives. Frontiers in Plant Science 11:770.
Jalili, S., Pandey, R. P., & Kurian, N. K. (2022). Recent Insights in Melanin Research: From Extraction to Immense Applications of The Pigment. Journal of Survey in Fisheries Sciences 8:140-146.
Liu, R., Meng, X., Mo, C., Wei, X., & Ma, A. (2022). Melanin of fungi: From classification to application. World Journal of Microbiology and Biotechnology 38:228.
Michael, H. S. R., Subiramanian, S. R., Thyagarajan, D., Mohammed, N. B., Saravanakumar, V. K., Govindaraj, M., Murugasamy Maheswari, K., Karthikeyan, N., & Kumar, C. R. (2023). Melanin biopolymers from microbial world with future perspectives—a review. Archives of Microbiology 205:306.
Nozella, N. L., Lima, J. V. M., de Oliveira, R. F., & Graeff, C. F. de O. (2023). Melanin/PEDOT:PSS blend as organic mixed ionic electronic conductor (OMIEC) for sustainable electronics. Materials Advances 4:4732-4743
Phiddian, E. (2022). Sunburn: How exactly does melanin shield you from UV—and what does it have to do with solar panels? Cosmos Magazine, Oct. 20, 2022
Pralea, I.-E., Moldovan, R.-C., Petrache, A.-M., Ilieş, M., Hegheş, S.-C., Ielciu, I., Nicoară, R., Moldovan, M., Radu, M., Uifălean, A., & Iuga, C.-A. (2019 August). From Extraction to Advanced Analytical Methods: The Challenges of Melanin Analysis. International Journal of Molecular Sciences 20 (16): 3943
Shahriarinour, M., Divsar, F., Mehdipour, A., Youseftabar-Miri, L., & Barkhordri, V. (2023). Antibacterial Properties of Cobalt Ferrite Magnetic Nanoparticles Loaded on Date Palm Pollen Against Multidrug-Resistant Bacteria. Arabian Journal for Science and Engineering 48:7315-7322
Singh, S., Nimse, S. B., Mathew, D. E., Dhimmar, A., Sahastrabudhe, H., Gajjar, A., Ghadge, V. A., Kumar, P., & Shinde, P. B. (2021). Microbial melanin: Recent advances in biosynthesis, extraction, characterization, and applications. Biotechnology Advances 53:107773
Solano, F. (2020). Photoprotection and skin pigmentation: Melanin-related molecules and some other new agents obtained from natural sources. Molecules 25:01537.
Vasanthabharathi, V., Lakshminarayanan, R., & Jayalakshmi, S. (2011). Melanin production from marine Streptomyces. African Journal of Biotechnology 10 (54): 11224-11234
Yuan, B., Yang, J., Li, L., Zhang, J., Wang, H. (2025) Infinite Organic Solid-Solution Semiconductors with Continuous Evolution in Film Morphology, Crystalline Lattice and Electrical Properties. Small January 16:32410159

We claim:

1. A method of producing melanin, said method comprising extracting said melanin from date palm pollen (DPP) from a species of the genus Phoenix.

2. The method of claim 1, wherein said DPP is from Phoenix dactylefera L.

3. The method of claim 1, wherein said extracting comprises ultrasound-assisted extraction of the melanin from the DPP.

4. The method of claim 1, wherein said extracting comprises:
(a) contacting said DPP with a strong alkali solution, thereby producing an alkaline extract solution that comprises solubilized extracted melanin.

5. The method of claim 4, further comprising:
(b) separating alkali soluble melanin from non-soluble components of the DPP;
(c) contacting the alkali soluble melanin with a strong acid solution, thereby precipitating the melanin; and
(d) treating the precipitated melanin with one or more organic solvent(s) to remove lipids and other residue, thereby producing purified melanin.

6. The method of claim 5, wherein step (b) comprises centrifuging the alkaline solution produced in step (a), thereby producing a supernatant that comprises the alkali soluble melanin.

7. The method of claim 5, wherein the organic solvent(s) in step (d) comprise chloroform, ether and/or petroleum ether.

8. An aqueous alkaline extract of DPP that comprises melanin.

9. Purified melanin, wherein said melanin is purified according to the method of claim 5.

10. A composition formulated for topical application, said composition comprising an aqueous alkaline extract of DPP or purified melanin from DPP according to claim 9.

11. The composition of claim 10, wherein said melanin exhibits photoprotective, antioxidant, antimicrobial, and/or anti-radiation properties when applied to the skin of an individual.

12. A method for treating a wound, comprising administering the composition according to claim 10 to a skin wound in an individual in need thereof, wherein healing of the wound is accelerated by said composition.

13. A method for improving the appearance of vitiligo, comprising administering the composition according to claim 10 to skin of an individual that has lost pigmentation, wherein said administering results in at least partial repigmentation of the skin to which the composition is applied.

14. A bioderived organic semiconductor, comprising DPP from a species of the genus Phoenix or purified melanin from DPP.

15. A melanin based electronic device, comprising the organic semiconductor according to claim 14.

16. A medical sensor, comprising the organic semiconductor according to claim 14.

17. An e-skin device, comprising the medical sensor according to claim 16.

18. A wafer for incorporation into an electronic device, comprising the organic semiconductor according to claim 14 blended with one or more polymer.

19. The wafer according to claim 18, wherein the one or more polymer comprises polydimethyl siloxane or poly(2,4-ethylenedioxythiophene) polystyrene sulfonate (PEDOT:PSS).

20. A method according to claim 1, wherein the amount of said melanin that is extracted from said DPP is at least about 5 times greater than the amount of melanin that is extracted from an equivalent weight of date fruit from the same species.
